(19) United States
(12) Patent Application Publication
(10) Pub. No.: US 2011/0077310 A1
(43) Pub. Date: Mar. 31, 2011

(54) HYDROPHILIC POLYURETHANE COATINGS

(75) Inventor: Juergen Koecher, Langenfeld (DE)

(73) Assignee: Bayer Material Science AG, Leverkusen (DE)

(21) Appl. No.: 12/994,665

(22) PCT Filed: May 19, 2009
(86) PCT No.: PCT/EP2009/003544

§ 371 Date: Nov. 24, 2010

(30) Foreign Application Priority Data
May 28, 2008 (DE) 10 2008 025 614.5

(51) Int. Cl. C09D 175/04; A61K 8/87

(52) U.S. Cl. 514/788; 524/590

(57) ABSTRACT

The present invention relates to the use of specific polyurethane coatings, wherein the polyurethane urea is terminated by a copolymer unit of polyethylene oxide and polypropylene oxide.

The present invention relates to the use of a coating composition in the form of a polyurethane dispersion in the production of hydrophilic coatings, in particular to the use of the coating composition in the coating of devices, in particular medical devices. In addition, the hydrophilic coating materials according to the invention can also be used to protect surfaces from condensation, to produce surfaces that are easy to clean or self-cleaning, and to reduce the uptake of dirt by such surfaces. The hydrophilic coating materials according to the invention are additionally capable of reducing or avoiding the formation of water spots on surfaces.

It is further possible with the polyurethane solutions according to the invention to produce hydrophilic surfaces which no longer become overgrown to a noteworthy extent with organisms that live in water (antifouling properties). Further fields of application of the coating materials according to the invention are applications in the printing industry, for cosmetic formulations as well as for systems for non-medical applications that release active ingredients.

The use of medical devices, for example catheters, can be greatly improved by providing them with hydrophilic surfaces. The insertion and displacement of urinary or blood vessel catheters is simplified because hydrophilic surfaces in contact with blood or urine adsorb a water film. As a result, friction of the catheter surface against the vessel walls is reduced, so that the catheter is easier to insert and move. Direct wetting of the devices before the operation can also be carried out in order to reduce friction through the formation of a homogeneous water film. The patients concerned have less pain, and the risk of damage to the vessel walls is thereby reduced. In addition, when catheters are used there is always the risk that blood clots will form. In this context, hydrophilic coatings are generally regarded as being helpful for antithrombogenic coatings.

Polyurethane coatings prepared from solutions or dispersions of corresponding polyurethanes are suitable in principle for the production of corresponding surfaces.

Thus, U.S. Pat. No. 5,589,563 describes the use of coatings having surface-modified end groups for polymers used in the biomedical field, which polymers can also be used to coat medical devices. The resulting coatings are produced from solutions or dispersions, and the polymeric coatings comprise different end groups which are selected from amines, fluorinated alkanols, polydimethylsiloxanes and amine-terminated polyethylene oxides. However, these polymers do not have satisfactory properties as coatings for medical devices, in particular in respect of the required hydrophilicity.

A disadvantage of aqueous dispersions as are described inter alia in U.S. Pat. No. 5,589,563 is additionally that the coatings are relatively rough owing to the size of the dispersed particles. In addition, the resulting coatings from aqueous dispersions are generally not sufficiently stable. There is therefore a need for hydrophilic coating systems which have outstanding hydrophilicity and at the same time have a relatively smooth surface and high stability.

Polyurethane solutions per se are known from the prior art but—with the exception of the polyurethane solutions according to U.S. Pat. No. 5,589,563 which have already been mentioned—have not been used in the coating of medical devices.

Thus, DE-A 22 21 798, for example, describes a process for the preparation of stable and light-resistant solutions of polyurethane ureas from prepolymers having terminal isocyanate groups and diamines in relatively non-polar solvents, wherein prepolymers of
a) substantially linear polyhydroxyl compounds having molecular weights of approximately from 500 to 5000,
b) optionally low molecular weight dihydroxy compounds and
c) aliphatic or cycloaliphatic diisocyanates, wherein the molar ratio of hydroxyl groups to isocyanate groups is approximately from 1:1.5 to 1:5,
   are reacted, in a solvent (mixture) of optionally chlorinated aromatic and/or chlorinated aliphatic hydrocarbons and primary, secondary and/or tertiary aliphatic and/or cycloaliphatic alcohols, with diamines as chain extender, wherein at least 80 mol % of the chain extender is 1,4-diaminocyclohexane having a cis/trans isomer ratio of from 10/90 to 60/40. These polyurethane urea solutions are used in the production of light-stable films and coatings.

Further, DE-A 22 52 280 describes a process for the coating of textile substrates by the reverse process with adhesive and top coats of solutions of aliphatic, segmented polyurethane elastomers containing polycarbonates.

Further, EP-A 0 125 466 describes a process for the multilayer reverse coating of textiles substrates, preferably in sheet form, in order to produce synthetic leather from at least one top-coat solution and at least one adhesive-coat solution based on polyurethanes.

None of these publications describes a hydrophilic polyurethane resin solution which is used for the purpose of coating medical devices and fulfils the demands defined hereinbefore.

Accordingly, it was an object of the present invention to provide compositions which are suitable for coating medical devices with hydrophilic surfaces. Because these surfaces are frequently used in contact with blood, the surfaces of these materials should also have good blood compatibility and, in particular, reduce the risk of blood clot formation. The resulting coatings should also be smooth and have high stability.

This invention provides coating compositions in the form of specific polyurethane solutions.

The polyurethane solutions to be used according to the invention comprise at least one polyurethane urea terminated by a copolymer unit of polyethylene oxide and polypropylene oxide.

It has been found according to the invention that compositions comprising these specific polyurethane ureas in solutions are outstandingly suitable for the production of coatings on medical devices, provide those devices with an excellent hydrophilic coating, form smooth surfaces, have high stability and at the same time reduce the risk of blood clot formation during treatment with the medical device.

Polyurethane ureas within the scope of the present invention are polymeric compounds comprising
(a) at least two urethane-group-containing structural repeating units having the following general structure

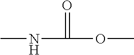

and
(b) at least one urethane-group-containing structural repeating unit

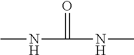

The coating compositions in the form of a solution that are to be used according to the invention are based on polyurethane ureas which have substantially no ionic modification. Within the scope of the present invention this is understood as meaning that the polyurethane ureas to be used according to the invention contain substantially no ionic groups, such as in particular no sulfonate, carboxylate, phosphate or phosphonate groups.

Within the scope of the present invention, the expression “substantially no ionic groups” is understood as meaning that the resulting coating of the polyurethane urea contains ionic groups in an amount of generally not more than 2.50 wt. %, in particular not more than 2.00 wt. %, preferably not more than 1.50 wt. %, particularly preferably not more than 1.00 wt. %, especially not more than 0.50 wt. %, more especially contains no ionic groups. It is particularly preferred for the polyurethane urea to contain no ionic groups because high concentrations of ions in organic solution have the result that the polymer is no longer sufficiently soluble and accordingly stable solutions cannot be obtained. If the polyurethane used according to the invention contains ionic groups, they are preferably carboxylates.

The coating compositions used in the form of solutions according to the invention preferably comprise polyurethanes which are preferably substantially linear molecules but can also be branched. In connection with the present invention, “substantially linear molecules” are understood as being systems that are readily pre-crosslinked and contain a polyol component having a mean hydroxyl functionality of preferably from 1.7 to 2.3, in particular from 1.8 to 2.2, particularly preferably from 1.9 to 2.1.

The number-average molecular weight of the polyurethane ureas that are preferably used according to the invention is preferably from 1000 to 200,000, particularly preferably from 5000 to 100,000. The number-average molecular weight is thereby measured against polystyrene as standard in dimethylacetamide at 30° C.

### Polyurethane Ureas

The coating systems based on polyurethane ureas that are to be used according to the invention are described in detail hereinbelow.

The polyurethane-containing coating compositions according to the invention in the form of a solution are prepared by reaction of chain-extension components which comprise at least one polycarbonate polyol component, at least one polyisocyanate component, at least one polyoxyalkylene ether component, at least one diamine and/or amino alcohol component and optionally a further polyol component.

The individual chain-extension components are described in detail hereinbelow.

### (a) Polycarbonate Polyol

The coating composition based on polyurethane urea in the form of a solution according to the invention comprises units based on at least one hydroxyl-group-containing polycarbonate.

For the introduction of units based on a hydroxyl-group-containing polycarbonate there are suitable in principle polyhydroxy compounds having a mean hydroxyl functionality of from 1.7 to 2.3, preferably from 1.8 to 2.2, particularly preferably from 1.9 to 2.1.

Suitable hydroxyl-group-containing polycarbonates are polycarbonates having a molecular weight, determined by the 014 number, of preferably from 400 to 6000 g/mol, particularly preferably from 500 to 5000 g/mol, especially from 600 to 3000 g/mol, which are obtainable, for example, by reaction of carbonic acid derivatives, such as diphenyl carbonate, dimethyl carbonate or phosgene, with polyols, preferably diols. There are suitable as such diols, for example, ethylene glycol, 1,2- and 1,3-propanediol, 1,3- and 1,4-butanediol, 1,6-hexanediol, 1,8-octanediol, neopentyl glycol, 1,4-bishydroxymethylcyclohexane, 2-methyl-1,3-propanediol, 2,2,4-trimethylpentane-1,3-diol, di-, tri- or tetra-ethylene glycol, dipropylene glycol, polypropylene glycols, dibutylene glycol, polybutylene glycols, bisphenol A, tetrabromobisphenol A, but also lactone-modified diols.

The diol component preferably contains from 40 to 100 wt. % hexanediol, preferably 1,6-hexanediol and/or hexanediol derivatives, preferably those which, as well as containing terminal OH groups, contain ether or ester groups, for example products obtained by reaction of 1 mole of hexanediol with at least 1 mole, preferably from 1 to 2 moles, of caprolactone or by etherification of hexanediol with itself to give di- or tri-hexylene glycol. Polyether polycarbonate diols can also be used. The hydroxyl polycarbonates should be substantially linear. They can, however, optionally be branched slightly by the incorporation of polyfunctional components, in particular low molecular weight polyols. Suitable for this purpose are, for example, glycerol, trimethylolpropane, 1,2,6-hexanetriol, 1,2,4-butanetriol, trimethylolpropane, pentaerythritol, quinitol, mannitol, sorbitol, methyl glycoside or 1,3,4,6-dianhydrohexite. Preference is given to polycarbonates based on 1,6-hexanediol as well as co-diols having a modifying action, such as, for example, 1,4-butanediol, or also on ε-caprolactone. Further preferred polycarbonate diols are those based on mixtures of 1,6-hexanediol and 1,4-butanediol.

The polycarbonate is preferably substantially linear and exhibits only slight three-dimensional crosslinking, so that polyurethanes having the above-mentioned specification are formed.

### (b) Polyisocyanate

The coating composition based on polyurethane urea according to the invention comprises as chain-extension component units based on at least one polyisocyanate.

There can be used as polyisocyanates (b) any aromatic, araliphatic, aliphatic and cycloaliphatic isocyanates known to the person skilled in the art having a mean NCO functionality ≧1, preferably ≧2, on their own or in arbitrary mixtures with one another, it being unimportant whether they have been prepared by phosgene or phosgene-free processes. They can also contain iminooxadiazinedione, isocyanurate, uretdione, urethane, allophanate, biuret, urea, oxadiazinetrione, oxazolidinone, acylurea and/or carbodiimide structures. The polyisocyanates can be used on their own or in arbitrary mixtures with one another.

Preference is given to the use of isocyanates from the group of the aliphatic or cycloaliphatic representatives, these preferably having a carbon skeletal structure (without the NCO groups that are present) of from 3 to 30, preferably from 4 to 20, carbon atoms.

Particularly preferred compounds of component (b) correspond to the above-mentioned type having aliphatically and/or cycloaliphatically bonded NCO groups, such as, for example, bis-(isocyanatoalkyl) ethers, bis- and tris-(isocyanatoalkyl)-benzenes, -toluenes and -xylenes, propane diisocyanates, butane diisocyanates, pentane diisocyanates, hexane diisocyanates (e.g. hexamethylene diisocyanate, HDI), heptane diisocyanates, octane diisocyanates, nonane diisocyanates (e.g. trimethyl-HDI (TMDI), generally in the form of a mixture of the 2,4,4- and 2,2,4-isomers), nonane triisocyanates (e.g. 4-isocyanatomethyl-1,8-octane diisocyanate), decane diisocyanates, decane triisocyanates, undecane diisocyanates, undecane triisocyanates, dodecane diisocyanates, dodecane triisocyanates, 1,3- and 1,4-bis-(isocyanatomethyl)cyclohexane (H₆XDI), 3-isocyanato-methyl-3,5,5-trimethylcyclohexyl isocyanate (isophorone diisocyanate, IPDI), bis-(4-isocyanatocyclohexyl)methane (H₁₂MDI) or bis(isocyanatomethyl)norbornane (NBDI).

Most particularly preferred compounds of component (b) are hexamethylene diisocyanate (HDI), trimethyl-HDI (TMDI), 2-methylpentane-1,5-diisocyanate (MPDI), isophorone diisocyanate (IPDI), 1,3- and 1,4-bis(isocyanatomethyl)cyclohexane (H₆XDI), bis(isocyanatomethyl)norbornane (NBDI), 3(4)-isocyanatomethyl-1-methyl-cyclohexylisocyanate (IMCI) and/or 4,4′-bis(isocyanatocyclohexyl)methane (H₁₂MDI) or mixtures of these isocyanates. Further examples are derivatives of the above diisocyanates having a uretdione, isocyanurate, urethane, allophanate, biuret, iminooxadiazinedione and/or oxadiazinetrione structure with more than two NCO groups.

The amount of constituent (b) in the coating composition to be used according to the invention is preferably from 1.0 to 3.5 mol, particularly preferably from 1.0 to 3.3 mol, especially from 1.0 to 3.0 mol, in each case based on constituent (a) of the coating composition to be used according to the invention.

### (c) Polyoxyalkylene Ethers

The polyurethane urea used in the present invention comprises as chain-extension component units based on a copolymer of polyethylene oxide and polypropylene oxide. These copolymer units are present in the polyurethane urea as end groups and effect hydrophilisation of the coating composition according to the invention.

Non-ionic hydrophilising compounds (c) are, for example, monohydric polyalkylene oxide polyether alcohols having in the statistical mean from 5 to 70, preferably from 7 to 55, ethylene oxide units per molecule, as are obtainable in a manner known per se by alkoxylation of suitable starter molecules (e.g. in Ullmanns Enzyklopädie der technischen Chemie, 4th Edition, Volume 19, Verlag Chemie, Weinheim p. 31-38).

Suitable starter molecules are, for example, saturated monoalcohols such as methanol, ethanol, n-propanol, isopropanol, n-butanol, isobutanol, sec-butanol, the isomeric pentanols, hexanols, octanols and nonanols, n-decanol, n-dodecanol, n-tetradecanol, n-hexadecanol, n-octadecanol, cyclohexanol, the isomeric methylcyclohexanols or hydroxymethylcyclohexane, 3-ethyl-3-hydroxymethyloxetan or tetrahydrofurfuryl alcohol, diethylene glycol monoalkyl ethers, such as, for example, diethylene glycol monobutyl ether, unsaturated alcohols such as allyl alcohol, 1,1-dimethylallyl alcohol or oleic alcohol, aromatic alcohols such as phenol, the isomeric cresols or methoxyphenols, araliphatic alcohols such as benzyl alcohol, anisic alcohol or cinnamic alcohol, secondary monoamines such as dimethylamine, diethylamine, dipropylamine, diisopropylamine, dibutylamine, bis-(2-ethylhexyl)-amine, N-methyl- and N-ethyl-cyclo-hexylamine or dicyclohexylamine as well as heterocyclic secondary amines such as morpholine, pyrrolidine, piperidine or 1H-pyrazole. Preferred starter molecules are saturated monoalcohols. Diethylene glycol monobutyl ether is particularly preferably used as the starter molecule.

The alkylene oxides ethylene oxide and propylene oxide can be used in the alkoxylation reaction in an arbitrary order or also in admixture.

The polyalkylene oxide polyether alcohols are mixed polyalkylene oxide polyethers of ethylene oxide and propylene oxide, the alkylene oxide units of which consist preferably of at least 30 mol %, particularly preferably of at least 40 mol %, ethylene oxide units. Preferred non-ionic compounds are monofunctional mixed polyalkylene oxide polyethers which contain at least 40 mol % ethylene oxide units and not more than 60 mol % propylene oxide units.

The mean molar weight of the polyoxyalkylene ether is preferably from 500 g/mol to 5000 g/mol, particularly preferably from 1000 g/mol to 4000 g/mol, especially from 1000 to 3000 g/mol.

The amount of constituent (c) in the coating composition to be used according to the invention is preferably from 0.01 to 0.5 mol, particularly preferably from 0.02 to 0.4 mol, especially from 0.04 to 0.3 mol, in each case based on constituent (a) of the coating composition to be used according to the invention.

It has been possible to demonstrate according to the invention that polyurethane ureas having end groups based on mixed polyoxyalkylene ethers of polyethylene oxide and polypropylene oxide are particularly suitable for producing coatings having high hydrophilicity. As is shown hereinbelow in comparison with polyurethane ureas terminated only by polyethylene oxide, the coatings according to the invention bring about a clearly small contact angle and are accordingly more hydrophilic.

### (d) Diamine or Amino Alcohol

The polyurethane urea solution according to the invention comprises as chain-extension component units that are based on at least one diamine or amino alcohol and act as so-called chain extenders (d).

Such chain extenders are, for example, di- or poly-amines as well as hydrazides, for example hydrazine, ethylenediamine, 1,2- and 1,3-diaminopropane, 1,4-diaminobutane, 1,6-diaminohexane, isophoronediamine, isomeric mixture of 2,2,4- and 2,4,4-trimethylhexamethylenediamine, 2-methylpentamethylenediamine, diethylenetriamine, 1,3- and 1,4-xylylenediamine, α,α,α′,α′-tetramethyl-1,3- and -1,4-xylylenediamine and 4,4′-diaminodicyclohexylmethane, dimethylethylenediamine, hydrazine, adipic acid dihydrazide, 1,4-bis(aminomethyl)cyclohexane, 4,4′-diamino-3,3′-dimethyldicyclohexyl-methane and other (C₁-C₄)-di- and tetra-alkyldicyclohexylmethanes, for example 4,4′-diamino-3,5-diethyl-3′,5′-diisopropyldicyclohexylmethane.

There come into consideration as diamines or amino alcohols generally low molecular weight diamines or amino alcohols which contain active hydrogen of different reactivity towards NCO groups, such as compounds that contain secondary amino groups in addition to a primary amino group or OH groups in addition to an amino group (primary or secondary). Examples thereof are primary and secondary amines, such as 3-amino-1-methylaminopropane, 3-amino-1-ethylaminopropane, 3-amino-1-cyclohexylamino-propane, 3-amino-1-methylaminobutane, also amino alcohols, such as N-aminoethyl-ethanolamine, ethanolamine, 3-aminopropanol, neopentanolamine and, particularly preferably, diethanolamine

Constituent (d) of the coating composition to be used according to the invention can be used in the preparation thereof as a chain extender.

The amount of constituent (d) in the solution of the coating composition according to the invention is preferably from 0.1 to 1.5 mol, particularly preferably from 0.2 to 1.3 mol, especially from 0.3 to 1.2 mol, in each case based on constituent (a) of the coating composition to be used according to the invention.

### (e) Polyols

In a further embodiment, the coating composition according to the invention in the form of a solution additionally comprises as chain-extension component units based on at least one further polyol.

The further low molecular weight polyols (e) used in the synthesis of the polyurethane ureas generally effect stiffening and/or branching of the polymer chain. The molecular weight is preferably from 62 to 500 g/mol, particularly preferably from 62 to 400 g/mol, especially from 62 to 200 g/mol.

Suitable polyols can contain aliphatic, alicyclic or aromatic groups. Examples which may be mentioned here include low molecular weight polyols having up to approximately 20 carbon atoms per molecule, such as, for example, ethylene glycol, diethylene glycol, triethylene glycol, 1,2-propanediol, 1,3-propanediol, 1,4-butanediol, 1,3-butylene glycol, cyclohexanediol, 1,4-cyclohexanedimethanol, 1,6-hexanediol, neopentyl glycol, hydroquinone dihydroxy ethyl ether, bisphenol A (2,2,-bis(4-hydroxyphenyl)propane), hydrogenated bisphenol A (2,2-bis(4-hydroxycyclohexyl)propane), as well as trimethylolpropane, glycerol or pentaerythritol and mixtures of these and optionally also further low molecular weight polyols. It is also possible to use ester diols, such as, for example, α-hydroxybutyl-ε-hydroxy-caproic acid ester, Ω-hydroxyhexyl-γ-hydroxy-butyric acid ester, adipic acid (β-hydroxyethyl)ester or terephthalic acid bis(β-hydroxy-ethyl)ester.

The amount of constituent (e) in the coating composition to be used according to the invention is preferably from 0.05 to 1.0 mol, particularly preferably from 0.05 to 0.5 mol, especially from 0.1 to 0.5 mol, in each case based on constituent (a) of the coating composition to be used according to the invention.

(f) Further Amine—and/or Hydroxy-Containing Structural Units (Chain-Extension Component)

The reaction of the isocyanate-containing component (b) with the hydroxy- or amine-functional compounds (a), (c), (d) and optionally (e) is usually carried out while maintaining a slight NCO excess relative to the reactive hydroxy or amine compounds. At the end point of the reaction, which is reached when a target viscosity is achieved, residues of active isocyanate still remain. These residues must be blocked so that a reaction with large polymer chains does not take place. Such a reaction leads to three-dimensional crosslinking and gelling of the batch. It is no longer possible to process such a coating solution. The batches usually contain large amounts of alcohols. The alcohols block the remaining isocyanate groups within several hours if the batch is left to stand or is stirred at room temperature.

If, however, the remaining residual isocyanate content is to be blocked quickly, the polyurethane urea coating compositions in the form of a solution that are provided according to the invention can also contain as chain-extension components monomers (f) which are in each case located at the ends of the polymer chains and close them off.

These structural components are derived on the one hand from monofunctional compounds reactive with NCO groups, such as monoamines, in particular monosecondary amines or monoalcohols. Examples which may be mentioned here include ethanol, n-butanol, ethylene glycol monobutyl ether, 2-ethylhexanol, 1-octanol, 1-dodecanol, 1-hexadecanol, methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine and suitable substituted derivatives thereof.

Because the structural units (f) are used in the coating composition according to the invention in the form of a solution substantially in order to destroy the NCO excess, the required amount is substantially dependent on the amount of the NCO excess and cannot generally be specified.

Preferably, these structural units are omitted during the synthesis. Isocyanate that has not yet reacted is preferably converted into terminal urethanes by the solvent alcohols present in very high concentrations.

### (g) Further Constituents

The solutions of the polyurethane urea coating composition provided according to the invention can additionally comprise further constituents and additives conventional for the intended purpose. An example thereof are pharmacological active ingredients, medicaments and additives which promote the release of pharmacological active ingredients (“drug-eluting additives”).

Pharmacological active ingredients or medicaments which can be used in the coatings according to the invention on medical devices and can accordingly be present in the solutions according to the invention are, for example, thromboresistant agents, antibiotic agents, antitumour agents, growth hormones, antiviral agents, antiangiogenic agents, angiogenic agents, antimitotic agents, antiinflammatory agents, cell-cycle-regulating agents, genetic agents, hormones, as well as their homologues, derivatives, fragments, pharmaceutical salts and combinations thereof.

Specific examples of such pharmacological active ingredients or medicaments accordingly include thromboresistant (non-thrombogenic) agents or other agents for suppressing an acute thrombosis, stenosis or late restenosis of the arteries, for example heparin, streptokinase, urokinase, tissue plasminogen activator, antithromboxane B₂ agents; anti-B thromboglobulin, prostaglandin E, aspirin, dipyridimol, antithromboxane A₂ agents, murine monoclonal antibody 7E3, triazolopyrimidine, ciprosten, hirudin, ticlopidine, nicorandil, etc. A growth factor can likewise be used as a medicament in order to suppress subintimal fibromuscular hyperplasia at the site of arterial stenosis, or any other desired inhibitor of cell growth at the stenosis site can be used.

The pharmacological active ingredient or medicament can also consist of a vasodilator in order to counteract vasospasm, for example an antispasmodic agent such as papaverin.

The medicament can be a vasoactive agent per se, such as calcium antagonists, or α- and β-adrenergic agonists or antagonists. In addition, the therapeutic agent can be a biological adhesive such as medical grade cyanoacrylate or fibrin, which is used, for example, to bond a tissue flap to the wall of a coronary artery.

The therapeutic agent can also be an antineoplastic agent such as 5-fluorouracil, preferably with a controlled-release carrier for the agent (e.g. for application of an antineoplastic agent that releases continuously in a controlled manner at a tumour site).

The therapeutic agent can be an antibiotic, preferably in combination with a controlled-release carrier for continuous release from the coating of a medical device at a localised source of infection within the body. Similarly, the therapeutic agent can comprise steroids for the purpose of suppressing inflammation in localised tissue or for other reasons.

### Specific Examples of Suitable Medicaments Include:

(a) heparin, heparin sulfate, hirudin, hyaluronic acid, chondroitin sulfate, dermatan sulfate, keratan sulfate, lytic agents, including urokinase and streptokinase, their homologues, analogues, fragments, derivatives and pharmaceutical salts thereof;
(b) antibiotic agents such as penicillins, cephalosporins, vacomycins, aminoglycosides, quinolones, polymxins, erythromycins; tetracyclines, chloramphenicols, clindamycins, lincomycins, sulfonamides, their homologues, analogues, derivatives, pharmaceutical salts and mixtures thereof;
(c) paclitaxel, docetaxel, immunosuppressants such as sirolimus or everolimus, alkylating agents including mechlorethamine, chlorambucil, cyclophosphamide, melphalan and ifosfamide; antimetabolites including methotrexate, 6-mercaptopurine, 5-fluorouracil and cytarabin; plant alkaloids including vinblastine; vincristine and etoposide; antibiotics including doxorubicin, daunomycin, bleomycin and mitomycin; nitrosurea including carmustin and lomustin; inorganic ions including cisplatin; biological reaction modifiers including interferon; angiostatins and endostatins; enzymes including asparaginase; and hormones including tamoxifen and flutamide, their homologues, analogues, fragments, derivatives, pharmaceutical salts and mixtures thereof;
(d) antiviral agents such as amantadine, rimantadine, rabavirin, idoxuridine, vidarabin, trifluridine, acyclovir, ganciclovir, zidovudine, phosphonoformates, interferons, their homologues, analogues, fragments, derivatives, pharmaceutical salts and mixtures thereof; and
(e) Antiinflammatory agents such as, for example, ibuprofen, dexamethasone or methylprednisolone.

In a preferred embodiment, the coating composition to be used according to the invention in the form of a solution comprises a polyurethane urea composed at least of
a) at least one polycarbonate polyol;
b) at least one polyisocyanate;
c) at least one monofunctional polyoxyalkylene ether; and
d) at least one diamine or amino alcohol.

In a further preferred embodiment of the present invention, the coating composition to be used according to the invention in the form of a solution comprises a polyurethane urea composed at least of
a) at least one polycarbonate polyol;
b) at least one polyisocyanate;
c) at least one monofunctional polyoxyalkylene ether;
d) at least one diamine or amino alcohol; and
e) at least one polyol.

In a further preferred embodiment of the present invention, the coating composition to be used according to the invention in the form of a solution comprises a polyurethane urea composed at least of
a) at least one polycarbonate polyol;
b) at least one polyisocyanate;
c) at least one monofunctional polyoxyalkylene ether;
d) at least one diamine or amino alcohol;
e) at least one polyol;
f) at least one further amine- and/or hydroxyl-containing structural unit.

The coating compositions to be used according to the invention in the form of solutions preferably comprise polyurethane ureas composed at least of
a) at least one polycarbonate polyol having a mean molar weight of from 400 g/mol to 6000 g/mol and a hydroxyl functionality of from 1.7 to 2.3, or mixtures of such polycarbonate polyols;
b) at least one aliphatic, cycloaliphatic or aromatic polyisocyanate, or mixtures of such polyisocyanates, in an amount, per mole of polycarbonate polyol, of from 1.0 to 3.5 mol;
c) at least one monofunctional polyoxyalkylene ether, or a mixture of such polyethers, having a mean molar weight of from 500 g/mol to 5000 g/mol in an amount, per mole of polycarbonate polyol, of from 0.01 to 0.5 mol;
d) at least one aliphatic or cycloaliphatic diamine or at least one amino alcohol as so-called chain extenders, or mixtures of such compounds, in an amount, per mole of polycarbonate polyol, of from 0.1 to 1.5 mol;
e) optionally one or more short-chained aliphatic polyols having a molar weight of from 62 g/mol to 500 g/mol in an amount, per mole of polycarbonate polyol, of from 0.05 to 1 mol; and
f) optionally amine- or OH-containing structural units which are located at the ends of the polymer chains and close them off.

Further preference is given according to the invention to the use, in the coating composition in the form of a solution, of polyurethane ureas composed at least of
a) at least one polycarbonate polyol having a mean molar weight of from 500 g/mol to 5000 g/mol and a hydroxyl functionality of from 1.8 to 2.2, or mixtures of such polycarbonate polyols;
b) at least one aliphatic, cycloaliphatic or aromatic polyisocyanate, or mixtures of such polyisocyanates, in an amount, per mole of polycarbonate polyol, of from 1.0 to 3.3 mol;
c) at least one monofunctional polyoxyalkylene ether, or a mixture of such polyethers, having a mean molar weight of from 1000 g/mol to 4000 g/mol in an amount, per mole of polycarbonate polyol, of from 0.02 to 0.4 mol;
d) at least one aliphatic or cycloaliphatic diamine or at least one amino alcohol as so-called chain extenders, or mixtures of such compounds, in an amount, per mole of polycarbonate polyol, of from 0.2 to 1.3 mol;
e) optionally one or more short-chained aliphatic polyols having a molar weight of from 62 g/mol to 400 g/mol in an amount, per mole of polycarbonate polyol, of from 0.05 to 0.5 mol; and
f) optionally amine- or OH-containing structural units which are located at the ends of the polymer chains and close them off.

Yet further preference is given according to the invention to the use, in the coating solution, of polyurethane ureas composed at least of
a) at least one polycarbonate polyol having a mean molar weight of from 600 g/mol to 3000 g/mol and a hydroxyl functionality of from 1.9 to 2.1, or mixtures of such polycarbonate polyols;
b) at least one aliphatic, cycloaliphatic or aromatic polyisocyanate, or mixtures of such polyisocyanates, in an amount, per mole of polycarbonate polyol, of from 1.0 to 3.0 mol;
c) at least one monofunctional polyoxyalkylene ether, or a mixture of such polyethers, having a mean molar weight of from 1000 g/mol to 3000 g/mol in an amount, per mole of polycarbonate polyol, of from 0.04 to 0.3 mol, a mixture of polyethylene oxide and polypropylene oxide being particularly preferred;
d) at least one aliphatic or cycloaliphatic diamine or at least one amino alcohol as so-called chain extenders, or mixtures of such compounds, in an amount, per mole of polycarbonate polyol, of from 0.3 to 1.2 mol; and
e) optionally one or more short-chained aliphatic polyols having a molar weight of from 62 g/mol to 400 g/mol in an amount, per mole of polycarbonate polyol, of from 0.1 to 0.5 mol.

In order to produce surfaces having antifouling properties, the coating compositions to be used according to the invention can comprise antifouling active ingredients known from the prior art. Their presence generally enhances the already outstanding antifouling properties of the surfaces produced with the coating compositions according to the invention themselves.

A coating composition to be used according to the invention in the form of a solution can be used to form a coating on a medical device.

The expression “medical device” is to be broadly interpreted within the scope of the present invention. Suitable non-limiting examples of medical devices (including instruments) are contact lenses; cannulas, catheters, for example urological catheters such as urinary catheters or uretral catheters, central venous catheters, venous catheters or inlet and outlet catheters; dilation balloons, catheters for angioplasty and biopsy, catheters used for insertion of a stent, a graft or a cava filter; balloon catheters or other expandable medical devices, endoscopes, laryngoscopes, tracheal devices such as endotracheal tubes, respiratory devices and other tracheal suction devices, bronchoalveolar lavage catheters, catheters used in coronary angioplasty, guide rods, inserters and the like, vessel grafts, pacemaker parts, cochlear implants, dental implant tubes for giving food, drainage tubes, and guide wires.

The coating solutions to be used according to the invention can additionally be used to produce protective coatings, for example for gloves, stents and other implants, extracorporeal blood tubes (blood guide tubes), membranes, for example for dialysis, blood filters, devices for assisting circulation, bandaging material for the care of wounds, urine bags and stoma bags. Also included are implants that contain a medically active agent, such as medically active agents for stents or for balloon surfaces or for contraceptives.

The medical device is usually formed from catheters, endoscopes, laryngoscopes, endotracheal tubes, feeding tubes, guide rods, stents and other implants.

Suitable substrates for the surface to be coated are many materials, such as metals, textiles, ceramics or plastics, the use of plastics being preferred for the production of medical devices.

It has been found according to the invention that medical devices having blood-compatible surfaces that are very hydrophilic, and therefore capable of sliding, can be produced by using aqueous, non-ionically stabilised polyurethane dispersions of the above-mentioned type for coating the medical devices. The above-described coating compositions are preferably obtained in the form of an organic solution and applied to the surface of the medical devices.

As well as being used as a coating for medical devices, the above-described coating compositions can also be used for further technical applications in the non-medical field.

Substrates for applications other than medical coatings are, for example, metals, plastics, ceramics, textiles, leather, wood, paper, coated surfaces of all the mentioned substrates, and glass. The coating materials can be applied directly to the substrate or alternatively to a base coat previously applied to the substrate.

Accordingly, the coatings produced according to the invention are used to protect surfaces from condensation, to produce surfaces that are easy to clean or self-cleaning. The hydrophilic coatings also reduce the uptake of dirt and prevent the formation of water spots. Possible applications in the external sector are, for example, window panes and skylights, glass façades or Plexiglass roofs. In the internal sector, such materials can be used to coat surfaces in the sanitary field. Further applications are the coating of optical glasses and lenses, such as, for example, spectacle lenses, binocular eyepiece and objective lenses and objective lenses for cameras, or of packaging materials, such as foodstuffs packaging, in order to avoid condensation or the formation of droplets by condensed water.

The coating materials to be used according to the invention are likewise suitable for application to surfaces in contact with water in order to prevent fouling. This effect is also known as an antifouling effect. A very important application of this antifouling effect is in the field of underwater paints for hulls. Hulls without antifouling properties very quickly become overgrown with marine organisms, which, owing to increased friction, leads to a reduction in the possible speed and to higher fuel consumption. The coating materials according to the invention reduce or prevent fouling with marine organisms and prevent the above-mentioned disadvantages of such fouling. Further applications within the field of antifouling coatings are articles for fishing, such as fishing nets, as well as all metal substrates that are used underwater, such as pipelines, drilling platforms, lock chambers and gates, etc. Hulls that have surfaces produced using the coating materials according to the invention, in particular beneath the water line, also have reduced frictional resistance so that ships having such properties either have a reduced fuel consumption or achieve higher speeds. This is of particular interest in the field of leisure craft and yacht building.

A further important field of use of the above-mentioned hydrophilic coating materials is the printing industry. Hydrophobic surfaces can be hydrophilised by the coatings according to the invention and can as a result be printed with polar printing inks or can be applied by means of ink-jet technology.

A further field of application of the hydrophilic coatings used according to the invention are formulations for cosmetic applications.

Active-ingredient-releasing systems based on the hydrophilic coating materials according to the invention are also conceivable outside medical technology, for example for applications in crop protection as a carrier for active ingredients. The coating as a whole can then be regarded as the active-ingredient-releasing system and can be used, for example, to coat seed (grains). As a result of the hydrophilic properties of the coating, the active ingredient that is present is able to emerge in the moist ground and develop its intended action without the germination capacity of the seed being impaired. In the dry state, however, the coating composition binds the active ingredient securely to the seed so that the active ingredient does not become detached, for example when the seed grain is injected into the ground by the sowing machine, as a result of which it could exert undesirable actions, for example on the fauna present (bees are endangered by insecticides which are to prevent the seed in the ground from being attacked by insects).

Particular preference is given to coating solutions composed of a mixture of polycarbonate polyols and a monofunctional polypropylene oxide-polyethylene oxide alcohol.

### Preparation of the Coating Solutions

Within the scope of the present invention it is particularly preferred for the coatings for medical devices to be produced from solutions of the coating composition described in detail hereinbefore.

It has been found according to the invention that the resulting coatings on medical devices differ depending on whether the above-described coating composition is prepared from a dispersion or a solution.

The coatings according to the invention on medical devices have advantages when they are obtained from solutions of the above-described coating compositions.

Without wishing to be bound to a theory, it is assumed according to the invention that, because of the particulate structure of the polyurethanes in aqueous dispersion, the film formation of the polymers is incomplete. The particle structures in the films are generally still detectable, for example by atomic force microscopy (AFM). Polyurethanes from solution yield smoother coatings. Because of the intimate entanglement of the polyurethane molecules in organic solution, the dried films also have greater tensile strength and are more resistant to storage in water.

The medical devices according to the invention can be coated with the hydrophilic polyurethane solutions by means of various processes. Suitable coating techniques for this purpose are, for example, knife coating, printing, transfer coating, spraying, spin coating or dipping.

The organic polyurethane solutions can be prepared by any desired processes.

The following procedure has been found to be preferred, however:

In order to prepare the polyurethane urea solutions to be used for coating according to the invention, the polycarbonate polyol, the polyisocyanate, the monofunctional polyether alcohol and optionally the polyol are preferably reacted with one another in the molten state or in solution until all the hydroxyl groups have been consumed.

The stoichiometry used thereby between the individual chain-extension components involved in the reaction is given by the ratios mentioned hereinbefore.

The reaction is carried out at a temperature of preferably from 60 to 110° C., particularly preferably from 75 to 110° C., especially from 90 to 110° C., temperatures of about 110° C. being preferred owing to the reaction rate. Higher temperatures can likewise be used, but there is then a risk, in individual cases and depending on the individual constituents used, that decomposition processes and discolourations will occur in the resulting polymer.

In the case of the prepolymer of isocyanate and all the components containing hydroxyl groups, reaction in the molten state is preferred, but there is a risk that the viscosities of the fully reacted mixtures will be too high. In such cases, it is also recommended to add solvents. However, not more than about 50 wt. % solvent should be present, if possible, because otherwise the dilution slows the reaction rate markedly.

In the reaction of isocyanate and the components containing hydroxyl groups, the reaction in the molten state can take place within a period of from 1 hour to 24 hours.

Additions of small amounts of solvent lead to a slowing down of the reaction, but the reaction times lie within the same times.

The sequence of addition or reaction of the individual constituents can differ from the sequence indicated hereinbefore. This can be advantageous in particular when the mechanical properties of the resulting coatings are to be changed. If, for example, all the components containing hydroxyl groups are reacted at the same time, a mixture of hard and soft segments is formed. If, for example, the low molecular weight polyol is added after the polycarbonate polyol component, defined blocks are obtained, which can be accompanied by different properties of the resulting coatings. The present invention is therefore not restricted to any sequence of addition or reaction of the individual constituents of the polyurethane coating.

Further solvent is then added, and the optionally dissolved chain-extending diamine or the dissolved chain-extending amino alcohol (chain-extension component (d)) is added.

The further addition of solvent is preferably carried out stepwise in order not to slow down the reaction unnecessarily, which would happen if all of the solvent was added, for example, at the beginning of the reaction. Furthermore, a high content of solvent at the beginning of the reaction means that the temperature, which is determined at least partly by the nature of the solvent, must be kept comparatively low. This also leads to a slowing down of the reaction.

When the target viscosity has been reached, the remaining residues of NCO can be blocked by a monofunctional aliphatic amine. The isocyanate groups that remain are preferably blocked by reaction with the alcohols contained in the solvent mixture.

Suitable solvents for the preparation and use of the polyurethane urea solutions according to the invention are all conceivable solvents and solvent mixtures, such as dimethylformamide, N-methylacetamide, tetramethylurea, N-methylpyrrolidone, aromatic solvents such as toluene, linear and cyclic esters, ethers, ketones and alcohols. Examples of esters and ketones are, for example, ethyl acetate, butyl acetate, acetone, γ-butyrolactone, methyl ethyl ketone and methyl isobutyl ketone.

Preference is given to mixtures of alcohols with toluene. Examples of alcohols which are used together with toluene are ethanol, n-propanol, isopropanol and 1-methoxy-2-propanol.

In general, the amount of solvent used in the reaction is such that approximately from 10 to 50 wt. % solutions, particularly preferably approximately from 15 to 45 wt. % solutions, particularly preferably approximately from 20 to 40 wt. % solutions, are obtained.

The solids content of the polyurethane solutions is generally from 5 to 60 wt. %, preferably from 10 to 40 wt. %. For coating tests, the polyurethane solutions can be diluted with toluene/alcohol mixtures as desired in order to allow the thickness of the coating to be variably adjusted. All concentrations from 1 to 60 wt. % are possible; concentrations in the range from 1 to 40 wt. % are preferred.

Any desired layer thicknesses can be achieved, such as, for example, from several 100 nm to several 100 μm, larger and smaller thicknesses also being possible within the scope of the present invention.

Further additives, such as, for example, antioxidants or pigments, can also be used. In addition, further additives, such as agents for improving handle, colourings, mattifying agents, UV stabilisers, light stabilisers, hydrophobising agents and/or flow improvers, can optionally be used.

Starting from these solutions, medical coatings are then produced by the processes described hereinbefore.

Many different substrates can be coated, such as metals, textiles, ceramics and plastics. Preference is given to the coating of medical devices manufactured from metals or plastics. Examples of metals which may be mentioned include: medical stainless steel and nickel-titanium alloys. There are many polymer materials of which the medical devices can be composed, for example polyamide; polystyrene; polycarbonate; polyethers; polyesters; polyvinyl acetate; natural and synthetic rubbers; block copolymers of styrene and unsaturated compounds such as ethylene, butylene and isoprene; polyethylene or copolymers of polyethylene and polypropylene; silicone; polyvinyl chloride (PVC) and polyurethanes. For the purpose of better adhesion of the hydrophilic polyurethane to the medical device, further suitable coatings can be applied as undercoat before the hydrophilic coating materials are applied.

The medical devices can be coated with the hydrophilic polyurethane dispersions by means of various processes. Suitable coating techniques are knife coating, printing, transfer coating, spraying, spin coating or dipping.

In addition to the hydrophilic properties for improving the sliding capacity, the layers produced with the coating compositions to be used according to the invention are also distinguished by high blood compatibility. As a result, it is also particularly advantageous to work with these coatings especially in contact with blood. Compared with polymers of the prior art, the materials have a reduced clotting tendency in contact with blood.

The advantages of the catheters obtained by the use according to the invention and provided with the hydrophilic polyurethane coatings are demonstrated in the following examples by means of comparison tests.

### EXAMPLES

The NCO content of the resins described in the examples and comparison examples was determined by titration according to DIN EN ISO 11909.

The solids contents were determined according to DIN-EN ISO 3251. 1 g of polyurethane dispersion was dried to constant weight (15-20 minutes) at 115° C. by means of an infra-red dryer.

The mean particle sizes of the polyurethane dispersions were measured with the aid of a High Performance Particle Sizer (HPPS 3.3) from Malvern Instruments.

Unless stated otherwise, the amounts in % are to be understood as being wt. % and are based on the resulting aqueous dispersion.

Viscosity measurements were carried out using a Physics MCR 51 rheometer from Anton Paar GmbH, Ostfildern, Germany.

### Substances and Abbreviations Used:

Desmophen® C2200: Polycarbonate polyol, OH number 56 mg KOH/g, number-average molecular weight 2000 g/mol (Bayer MaterialScience AG, Leverkusen, DE)
Desmophen® C1200: Polycarbonate polyol, OH number 56 mg KOH/g, number-average molecular weight 2000 g/mol (Bayer MaterialScience AG, Leverkusen, DE)
Desmophen® XP 2613 Polycarbonate polyol, OH number 56 mg KOH/g, number-average molecular weight 2000 g/mol (Bayer MaterialScience AG, Leverkusen, DE)
PolyTHF^{v} 2000: Polytetramethylene glycol polyol, OH number 56 mg KOH/g, number-average molecular weight 2000 g/mol (BASF AG, Ludwigshafen, DE)
Polyether® LB 25: (monofunctional polyether based on ethylene oxide/propylene oxide, number-average molecular weight 2250 g/mol, OH number 25 mg KOH/g (Bayer MaterialScience AG, Leverkusen, DE)

### Example 1

### Preparation of a Polyurethane Urea Solution According to the Invention

198.6 g of Desmophen® C 2200, 23.0 g of LB 25 and 47.8 g of 4,4′-bis(isocyanatocyclohexyl)methane (H₁₂MDI) were reacted at 110° C. in the molten state to a constant NCO content of 2.4%. The mixture was allowed to cool and was diluted with 350.0 g of toluene and 200 g of isopropanol. A solution of 12.5 g of isophoronediamine in 95.0 g of 1-methoxy-2-propanol was added at room temperature. When the molecular weight build-up was complete and the desired viscosity range had been reached (checking by measuring the viscosity of a withdrawn sample using the above-mentioned rheometer), stirring was carried out for a further 4 hours in order to block the residual isocyanate content with isopropanol. There were obtained 927 g of a 30.4% polyurethane urea solution in toluene/isopropanol/1-methoxy-2-propanol having a viscosity of 19,600 mPas at 22° C.

### Example 2

### Preparation of a Polyurethane Urea Solution According to the Invention

195.4 g of Desmophen® C 2200, 30.0 g of LB 25 and 47.8,g of 4,4′-bis(isocyanatocyclohexyl)methane (H₁₂MDI) were reacted at 110° C. to a constant NCO content of 2.3%. The mixture was allowed to cool and was diluted with 350.0 g of toluene and 200 g of isopropanol. A solution of 12.7 g of isophoronediamine in 94.0 g of 1-methoxy-2-propanol was added at room temperature. When the molecular weight build-up was complete and the desired viscosity range had been reached, stirring was carried out for a further 4 hours in order to block the residual isocyanate content with isopropanol. There were obtained 930 g of a 30.7% polyurethane urea solution in toluene/isopropanol/1-methoxy-2-propanol having a viscosity of 38,600 mPas at 22° C.

### Example 3

### Preparation of a Polyurethane Urea Solution According to the Invention

195.4 g of Desmophen® XP 2613, 30.0 g of LB 25 and 47.8 g of 4,4′-bis(isocyanatocyclohexyl)methane (H₁₂MDI) were reacted at 110° C. to a constant NCO content of 2.4%. The mixture was allowed to cool and was diluted with 350.0 g of toluene and 200 g of isopropanol. A solution of 12.7 g of isophoronediamine in 95.0 g of 1-methoxy-2-propanol was added at room temperature. When the molecular weight build-up was complete and the desired viscosity range had been reached, stirring was carried out for a further 4 hours in order to block the residual isocyanate content with isopropanol. There were obtained 931 g of a 30.7% polyurethane urea solution in toluene/isopropanol/1-methoxy-2-propanol having a viscosity of 26,500 mPas at 22° C.

### Example 4

### Preparation of a Polyurethane Urea Solution According to the Invention

198.6 g of Desmophen® C 1200, 23.0 g of LB 25 and 47.8 g of 4,4′-bis(isocyanatocyclo-hexyl)methane (H₁₂MDI) were reacted at 110° C. to a constant NCO content of 2.4%. The mixture was allowed to cool and was diluted with 350.0 g of toluene and 200 g of isopropanol. A solution of 13.2 g of isophoronediamine in 100.0 g of 1-methoxy-2-propanol was added at room temperature. When the molecular weight build-up was complete and the desired viscosity range had been reached, stirring was carried out for a further 4 hours in order to block the residual isocyanate content with isopropanol. There were obtained 933 g of a 30.3% polyurethane urea solution in toluene/isopropanol/1-methoxy-2-propanol having a viscosity of 17,800 mPas at 22° C.

### Example 5

### Preparation of a Polyurethane Urea Solution According to the Invention

195.4 g of Desmophen® C 1200, 30.0 g of LB 25 and 47.8 g of 4,4′-bis(isocyanatocyclohexyl)methane (H₁₂MDI) were reacted at 110° C. to a constant NCO content of 2.4%. The mixture was allowed to cool and was diluted with 350.0 g of toluene and 200 g of isopropanol. A solution of 11.8 g of isophoronediamine in 94.0 g of 1-methoxy-2-propanol was added at room temperature. When the molecular weight build-up was complete and the desired viscosity range had been reached, stirring was carried out for a further 4 hours in order to block the residual isocyanate content with isopropanol. There were obtained 931 g of a 30.7% polyurethane urea solution in toluene/isopropanol/1-methoxy-2-propanol having a viscosity of 23,700 mPas at 22° C.

### Example 6

### Preparation of a Polyurethane Urea Solution as Comparison Product to Example 1 According to the Invention. Desmophen® C2200 is replaced by PolyTHF 2000

194.0 g of PoIyTHF 2000, 22.6 g of LB 25 and 47.8 g of 4,4′-bis(isocyanatocyclohexyl)-methane (H₁₂MDI) were reacted at 110° C. to a constant NCO content of 2.3%. The mixture was allowed to cool and was diluted with 350.0 g of toluene and 200 g of isopropanol. A solution of 12.1 g of isophoronediamine in 89.0 g of 1-methoxy-2-propanol was added at room temperature. When the molecular weight build-up was complete and the desired viscosity range had been reached, stirring was carried out for a further 4 hours in order to block the residual isocyanate content with isopropanol. There were obtained 916 g of a 30.2% polyurethane urea solution in toluene/isopropanol/1-methoxy-2-propanol having a viscosity of 15,200 mPas at 22° C.

### Example 7

### Preparation of a Polyurethane Urea Solution as Comparison Product to Example 2 According to the Invention. Desmophen® C2200 is Replaced by PolyTHF 2000

190.6 g of PolyTHF 2000, 30.0 g of LB 25 and 47.8 g of 4,4′-bis(isocyanatocyclohexyl)-methane (H₁₂MDI) were reacted at 110° C. to a constant NCO content of 2.3%. The mixture was allowed to cool and was diluted with 350.0 g of toluene and 200 g of isopropanol. A solution of 12.1 g of isophoronediamine in 89.0 g of 1-methoxy-2-propanol was added at room temperature. When the molecular weight build-up was complete and the desired viscosity range had been reached, stirring was carried out for a further 4 hours in order to block the residual isocyanate content with isopropanol. There were obtained 919 g of a 30.5% polyurethane urea solution in toluene/isopropanol/1-methoxy-2-propanol having a viscosity of 21,000 mPas at 22° C.

### Example 8

### Production of Coatings and Measurement of the Static Contact Angle

The coatings for measurement of the static contact angle were produced by means of a spin coater (RC5 Gyrset 5, Karl Siiss, Garching, Germany) on glass slides having a size of 25×75 mm. To this end, a slide was clamped on the sample plate of the spin coater and covered homogeneously with approximately from 2.5 to 3 g of organic 15% polyurethane solution. All the organic polyurethane solutions were diluted to a polymer content of 15 wt. % with a solvent mixture of 65 wt. % toluene and 35 wt. % isopropanol. A homogeneous coating was obtained by rotating the sample plate for 20 seconds at 1300 revolutions per minute, which coating was dried for one hour at 100° C. and then for 24 hours at 50° C. The resulting coated slides were immediately subjected to a contact angle measurement.

A static contact angle measurement was carried out on the resulting coatings on the slides. 10 drops of Millipore water were applied to the sample by means of a video-based contact angle measuring device OCA20 from Dataphysics with computer-controlled syringes, and their static wetting angle was measured. The static charge (if present) on the sample surface was removed beforehand by means of an antistatic hair-dryer.

TABLE 1
Static contact angle measurements

| PU FILM | CONTACT ANGLE [°] |
|---|---|
| Example 1 | 32 |
| Example 2 | 21 |
| Example 3 | 38 |
| Example 4 | 25 |
| Example 5 | 25 |
| Example 6 | 83 |
| (comparison example) | |
| Example 7 | 82 |
| (comparison example) | |

As Table 1 shows, the polycarbonate-containing coatings of Examples 1 to 5 produce extremely hydrophilic coatings with static contact angles 40° . The polyTHF-containing coatings 7 to 9, on the other hand, are substantially more non-polar, although the compositions of these coatings are otherwise identical with those of Examples 1 and 2.

### Example 9

This comparison example describes the synthesis of a polyurethane urea polymer which, instead of containing the mixed monofunctional polyethylene-polypropylene oxide alcohol LB 25, contains the same molar amount of a pure monofunctional polyethylene oxide alcohol. The polymer is identical with that of Example 1, except that it contains a different terminal group. The synthesis in toluene and alcohols as described in Examples 1 to 7 is not possible when using this alcohol. The synthesis is therefore carried out in pure dimethylformamide (DMF).

198.6 g of Desmophen® C 2200, 20.4 g of polyethylene glycol 2000 monomethyl ether (source: Fluka, Article No. 81321) and 47.8 g of 4,4′-bis(isocyanatocyclohexyl)methane (H₁₂MDI) were reacted at 110° C. in the molten state to a constant NCO content of 2.4%. The mixture was allowed to cool and was diluted with 550 g of DMF. A solution of 10.5 g of isophoronediamine in 100 g of DMF was added at room temperature. When the molecular weight build-up was complete and the desired viscosity range had been reached (checking by measuring the viscosity of a withdrawn sample using the above-mentioned rheometer), 1.0 g of n-butylamine was added in order to block the remaining small isocyanate content. There were obtained 927 g of a 29.8% polyurethane urea solution in dimethylformamide having a viscosity of 22,700 mPas at 23° C.

### Example 10

### Synthesis of a Polyurethane Urea Polymer According to the Invention in DMF as Solvent

The polymer is identical with that of Example 1 but was prepared in DMF in order to be able to compare its physical properties with the polymer of Example 9.

198.6 g of Desmophen® C 2200, 23.0 g of LB 25 and 47.8 g of 4,4′-bis(isocyanatocyclohexyl)methane (H₁₂MDI) were reacted at 110° C. in the molten state to a constant NCO content of 2.4%. The mixture was allowed to cool and was diluted with 550 g of DMF. A solution of 10.5 g of isophoronediamine in 100 g of 1-methoxy-2-propanol was added at room temperature. When the molecular weight build-up was complete and the desired viscosity range had been reached (checking by measuring the viscosity of a withdrawn sample using the above-mentioned rheometer), 0.5 g of n-butylamine was added in order to block the remaining small isocyanate content with isopropanol. There were obtained 930 g of a 30.6% polyurethane urea solution in DMF having a viscosity of 16,800 mPas at 23° C.

### Example 11

As described in Example 8, films were produced on glass using the polyurethane solutions of Examples 9 and 10 and the static contact angle was measured.

TABLE 2
Static contact angle in dependence on
the monofunctional polyether used

| PU FILM | Contact angle [°] |
|---|---|
| Example 9 (comparison example) | 55 |
| Example 10 (example according to the | 36 |
| invention) | |

The film of Example 10 produced with the mixed (polyethylene oxide/polypropylene oxide) monofunctional polyether alcohol exhibits a markedly lower static contact angle of 36° compared with that of the film according to Example 9)(55°, which contains pure polyethylene oxide units.

### Example 12

Synthesis of a polyurethane according to the invention in organic solution. This product was compared with the polyurethane of Example 13 which was prepared in a corresponding manner in aqueous dispersion (see Example 14).

277.2 g of Desmophen C 2200, 33.1 g of LB 25, 6.7 g of neopentyl glycol, 71.3 g of 4,4′-bis(isocyanatocyclohexyl)methane (H₁₂MDI) and 11.9 g of isophorone diisocyanate were reacted at 110° C. to a constant NCO content of 2.4%. The mixture was allowed to cool and was diluted with 500.0 g of toluene and 350.0 g of isopropanol. A solution of 6.2 g of isophoronediamine in 186.0 g of 1-methoxy-2-propanol was added at room temperature. When the molecular weight build-up was complete and the desired viscosity range had been reached, stirring was carried out for a further 4 hours in order to block the residual isocyanate content with isopropanol. There were obtained 1442 g of a 28.6% polyurethane urea solution in toluene/isopropanol/1-methoxy-2-propanol having a viscosity of 17,500 mPas at 23° C.

### Example 13

Synthesis of the polyurethane of Example 12 in aqueous dispersion. It consists of the same polymer as described in Example 12. The two polymers are compared with one another in Example 14.

277.2 g of Desmophen® C 2200, 33.1 g of Polyether LB 25 and 6.7 g of neopentyl glycol were placed in a reaction vessel at 65° C. and homogenised for 5 minutes by stirring. To this mixture there were added at 65° C., in the course of one minute, first 71.3 g of 4,4′-bis(isocyanatocyclohexyl)methane (H₁₂MDI) and then 11.9 g of isophorone diisocyanate. The mixture was heated at 110° C. until a constant NCO value of 2.4% had been achieved. The finished prepolymer was dissolved at 50° C. in 711 g of acetone, and then a solution of 4.8 g of ethylenediamine in 16 g of water was metered in at 40° C. in the course of 10 minutes. The after-stirring time was 5 minutes. Dispersion was then carried out in the course of 15 minutes by addition of 590 g of water. The solvent was then removed by distillation in vacuo. A storage-stable polyurethane dispersion having a solids content of 40.7% and a mean particle size of 136 nm was obtained. The pH value of this dispersion was 6.7.

### Example 14

The two coatings of Examples 12 and 13 were applied to release paper with a 200 μm knife. The coating of Example 12 was applied in undiluted form; 2 wt. % of a thickener (Borchi Gel® A LA, Borchers, Langenfeld, Germany) were added to the aqueous dispersion prior to production of the film, and homogenisation was carried out by stirring for 30 minutes at RT. The moist films were dried for 15 minutes at 100° C.

Tensile strength and elongation at break were measured in the dry state and after the films had been watered for 24 hours. The tests were carried out according to DIN 53504.

TABLE 3
Comparison of the tensile strength results for polyurethanes
from organic solution and aqueous dispersion

| | Stress | Stress | Elongation at | Elongation at |
|---|---|---|---|---|
| | at break | at break | break | break |
| | (N/mm²) | (N/mm²) | (%) | (%) |
| Film | dry film | 24 h water | dry film | 24 h water |
| Example 12 | 25.3 | 24.9 | 700 | 700 |
| Example 13 | 24.8 | 18.3 | 550 | 450 |

The results in the table show that the stress at break of the dried films coincides, within the scope of experimental accuracy, for both polyurethanes, regardless of whether they were prepared as a solution or as an aqueous dispersion. The film of Example 12 produced from organic solution has higher elasticity, however (700% elongation at break compared with 550% for the polymer from aqueous dipersion). In addition, the stress at break and the elongation at break do not change within the scope of measuring accuracy for the film produced from organic solution, while there is a marked fall in the stress at break and elongation at break of the film produced from aqueous dispersion.

1-23. (canceled)

24. Use of a coating composition in the form of a solution containing at least one polyurethane urea which is terminated by a copolymer unit of polyethylene oxide and polypropylene oxide in the coating of substrates.

25. Use according to claim 24, characterised in that the polyurethane urea contains units based on at least one hydroxyl-group-containing polycarbonate.

26. Use according to claim 24, characterised in that the polyurethane urea contains units based on aliphatic or cycloaliphatic polyisocyanates.

27. Use according to claim 24, characterised in that the polyurethane urea contains units based on at least one polyol.

28. Use according to claim 24, characterised in that the polyurethane urea contains units based on at least one diamine or amino alcohol.

29. Use according to claim 24, characterised in that the polyurethane urea contains units based on further hydroxyl- and/or amine-containing chain-extension components.

30. Use according to claim 24, characterised in that the polyurethane urea is composed at least of the following chain-extension components
a) at least one polycarbonate polyol;
b) at least one polyisocyanate;
c) at least one monofunctional polyoxyalkylene ether; and
d) at least one diamine or amino alcohol.

31. Use according to claim 24, characterised in that the polyurethane urea additionally contains chain-extension components of
e) at least one polyol.

32. Use according to claim 24, characterised in that the polyurethane urea is composed at least of the following chain-extension components:
a) at least one polycarbonate polyol having a mean molar weight of from 400 g/mol to 6000 g/mol and a hydroxyl functionality of from 1.7 to 2.3, or mixtures of such polycarbonate polyols;
b) at least one aliphatic, cycloaliphatic or aromatic polyisocyanate, or mixtures of such polyisocyanates, in an amount, per mole of polycarbonate polyol, of from 1.0 to 3.5 mol;
c) at least one monofunctional polyoxyalkylene ether, or a mixture of such polyethers, having a mean molar weight of from 500 g/mol to 5000 g/mol in an amount, per mole of polycarbonate polyol, of from 0.01 to 0.5 mol;
d) at least one aliphatic or cycloaliphatic diamine or at least one amino alcohol as so-called chain extenders, or mixtures of such compounds, in an amount, per mole of polycarbonate polyol, of from 0.1 to 1.5 mol; and
e) optionally one or more short-chained aliphatic polyols having a molar weight of from 62 g/mol to 500 g/mol in an amount, per mole of polycarbonate polyol, of from 0.05 to 1.0 mol.

33. Use according to claim 24, comprising the steps
A) preparation of a coating composition by:
(I) reacting the polycarbonate polyol, the polyisocyanate and the monofunctional polyoxyalkylene ether and optionally the polyol in the molten state or in the presence of a solvent in solution, until all the hydroxyl groups have been consumed;
(II) adding further solvent and optionally adding the dissolved diamine or the optionally dissolved amino alcohol; and
(III) optionally blocking the residues of NCO groups still present after the target viscosity has been reached, by means of a monofunctional aliphatic amine, and
B) coating a substrate with the coating composition obtained according to (A).

34. Use according to claim 33, characterised in that the solvent is selected from the group consisting of dimethylformamide, N-methylacetamide, tetramethylurea, N-methylpyrrolidone, γ-butyrolactone, aromatic solvents, linear and cyclic esters, ethers, ketones, alcohols and mixtures thereof.

35. Use according to claim 33, characterised in that the solids content of the polyurethane solution is from 5 to 60 wt. %.

36. Use according to claim 24 in the coating of at least one medical device.

37. Use according to claim 24 in the coating of technical substrates in the non-medical field.

38. Use according to claim 24 in the production of surfaces that are easy to clean or self-cleaning.

39. Use according to claim 24 in the coating of glass and optical glasses and lenses.

40. Use according to claim 24 in the coating of substrates in the sanitary field.

41. Use according to claim 24 in the coating of packaging materials.

42. Use according to claim 24 for reducing fouling of the coated surfaces.

43. Use according to claim 24 in the coating of over- and under-water substrates in order to reduce the frictional resistance of the substrates towards water.

44. Use according to claim 24 to prepare substrates for printing.

45. Use according to claim 24 in the production of formulations for cosmetic applications.

46. Use according to claim 24 in the production of active-ingredient-releasing systems for coating seeds.
